(19) United States
(12) Patent Application Publication
(10) Pub. No.: US 2007/0032547 A1
(43) Pub. Date: Feb. 8, 2007

(54) Method and device for treating migraine, tension-type and post-traumatic headache, atypical facial pain, and cervical muscle hyperactivity

(76) Inventors: Mark Friedman, Scarsdale, NY (US); Richard Casey, Indianapollis, IN (US)

Correspondence Address: Evelyn M. Sommer, Suite 825, 250 Park Avenue, New York, NY, 10022, US

(21) Appl. No.: 11/196,831

(22) Filed: Aug. 4, 2005

(51) Int. Cl. A61N 1/39; A61K 31/24

(52) U.S. Cl. 514/534; 514/536; 607/1

(57) ABSTRACT

A new non-invasive, non-toxic, non-sedating method for treatment migraine, tension and post-traumatic headache, atypical facial pain, and cervical muscle hyperactivity (spasm) is presented. This method comprises the application of cold, through the use of a portable thermoelectric cooler, to an intra-oral area of tenderness which has been found to be associated with the above conditions. New portable thermoelectric cooler devices for use with the intended treatment methods are also presented. In one embodiment, the thermoelectric cooling units are directly housed in the U-shaped mouthpiece. In another embodiment, water is chilled thermoelectrically in a container and circulated through the U-shaped mouthpiece. Alleviation of pain is noted within a short period of the onset of the treatment. Repeated treatment even during pain-free periods offers protracted benefits, i.e., prevention or amelioration of the headaches or other conditions.

The present invention relates to a new method for treatment of migraine, tension and post-traumatic headache, atypical facial pain, and cervical muscle hyperactivity (spasm). More particularly, the present invention relates to a new method for treatment of the above conditions through the use of a portable thermoelectric cooler for applying cold to an area of intra-oral tenderness which has been found to be associated with the above conditions. The invention further relates to a new portable thermoelectric cooler device for use with the intended treatment method.

The method is non-invasive, non-toxic, and non-sedating.

The method of the invention comprises the application of a portable thermoelectric cooler (0-10° C.) to an area of intra-oral tenderness which has been found to be associated with the above conditions. This zone of tenderness was observed by one of the inventors to be in the area of the plexus formed by the posterior and middle superior alveolar branches of the ipsilateral maxillary nerve and is bilateral when the symptoms are bilateral.

The thermoelectric cooler is applied indirectly to an accessible segment of the maxillary nerve (V2), a division of the trigeminal nerve. This treatment produces a marked decrease or elimination of the intra-oral maxillary tenderness and, with repeated applications, a decrease in frequency and severity of headaches. When the patient is symptomatic, headache, facial pain, and cervical spasm relief occurs often in a matter of minutes. If the pain returns, or to provide more long lasting relief, the procedure is repeated.

Headaches can be divided into two main groups: primary and secondary. Primary headaches include migraines, tension-type, and cluster headache. Secondary headaches include a wide variety of disorders, including stroke, hypertension, intra-cranial bleeds, brain tumors, and post-traumatic headache.

Tension-type headache (new International Headache Society classification), formerly referred to as muscle contraction headache, is by far the most common headache. This condition is divided into two major categories, episodic tension-type headache and chronic tension type headache. The former is the familiar ailment that virtually everybody experiences occasionally. The latter condition may be related to migraine or post-traumatic headache. It is characterized by daily or almost daily discomfort often with superimposed migrainous events at varying frequencies, and may be the most common problem seen at headache centers. It is often referred to as chronic daily headache. Patients complain of daily headache, which comprises dull to moderate discomfort most of the time, except for occasions of varying frequency when the headache becomes severe. At such time, the pain may be associated with typical migrainous symptoms such as nausea, and vomiting, photophobia, and other descriptors of migraine. Patients usually complain of pain and tightness in the frontal, temporal and/or occipital areas. Pain in the latter area may indicate cervical involvement. The definition of “tension-type” headache is confusing, because the relationship of tension to the headache has not been established. Most authorities believe that psychological factors, including stress, cause muscle tension which produces the headache. Many of these patients exhibit significant cervical muscle spasm, which can refer or exaggerate as tension-type headache. Other factors such as postural deformities or poor work habits can also contribute to the headache directly or by causing cervical or craniofacial muscle spasm, thereby contributing indirectly to the tension-type headache.

Treatment for headache includes physical therapy, biofeedback, chiropractic, counseling, and medication. The latter includes analgesics, muscle relaxants, tranquilizers, and antidepressants. Combination drugs are widely used. Many of these patients tend to abuse a wide variety of medications, including both prescription and non-prescription drugs. Overuse often leads to an analgesic rebound headache or the worsening of the head pain caused by the frequent and excessive use of analgesics and other medications.

Migraine is the most common headache causing patients to consult a physician. Based largely on data drawn from the American Migraine Study, in which over 20,000 respondents returned questionnaires mailed to their households, the results indicate migraine occurs in 17.6% of females and 6% of males in the United States. Considering this incidence, the economics of migraine in time lost from work, inefficiency, etc. is substantial. Effective treatment increases the patient's ability to live a normal and productive life. In addition to pain, the symptoms most commonly associated with migraine include nausea and vomiting, photophobia, phonophobia, anorexia, pallor, and a desire to lie down. If symptoms are preceded by or associated with visual symptoms such as flashing lights, black spots or partial visual field loss, the migraine is classified as classic, as opposed to the previously described common migraine.

Multiple humoral agents have been postulated as being the major factor in migraine. These include serotonin, histamine, prostaglandins, platelet factors, endorphins, and vasoactive neuropeptides. The etiology of migraine has been studied by many investigators. Present research no longer supports the vasodilator/vasoconstrictor mechanism of vascular headache, i.e., arterial dilation causes pain and constriction equals relief. Research has now implicated a sterile inflammation, possibly occurring in the dura mater, as the causative factor for vascular head pain. An unknown trigger activates perivascular trigeminal axons, which release vasoactive neuropeptides (substance P, calcitonin gene-related peptide, etc.). These agents produce the local inflammation i.e., vasodilation, plasma extravasation, mast cell degranulation which cause transmission of impulses to the brain stem and higher centers which in turn register as head pain (Moskowitz M A, Trends in Pharmacological Sciences, August 1992).

Migraine therapy is either prophylactic or symptomatic. Prophylactic medication may be selected for a patient having two to four or more headaches per month, if they are severe enough to interfere with daily activities. Beta blockers such as propranolol (Inderal) are the most commonly used. Other medications frequently used include serotonin antagonists such as methysergide maleate (Sansert), calcium channel blockers (Verapamil), amytryptyline (Elavil), and ergotamine preparations with belladona alkaloids and phenobarbital. All of these medications have significant side effects including sedation, loss of energy and drive, dry mouth, constipation, weight gain, and gastrointestinal cramping and distress. For symptomatic treatment, ergotamine with caffeine (cafergot) is commonly used. Other medications employed for treating migraine include isometheptene mucate (Midrin), NSAID's (Motrin, Naprosyn, etc.), dihydroergotamine and the newer triptans, such as sumatriptan (Imitrex) and etc. When narcotics, such as Fiorinal with codeine are used frequently, additional hazards, including the considerable potential for rebound headaches and habituation are encountered.

Post-traumatic headache is a secondary headache that arises after head injury. Often part of the post-traumatic syndrome, it usually follows mild-to-moderate closed head injury even without loss of consciousness. In addition to headache, symptoms include neck pain, depression, irritability, memory impairment, dizziness or vertigo, and difficulty in concentrating. Head injuries have three major causes—motor vehicle, occupational, and recreational accidents. Trauma may exacerbate a pre-existing headache disorder, such as migraine, cluster or tension-type headache. It may also be an initiating event, producing the first of many headache attacks. The essence of the diagnosis of post-traumatic headache is that cranial trauma initiated the headache, which may be accompanied by various aspects of post-traumatic syndrome. Post-traumatic headache is almost invariably accompanied by cervical dysfunction. This latter condition, often induced by whiplash, refers to the extension, flexion, and lateral motions of the neck that follow impact, with or without direct trauma to the head. Post-traumatic headache usually resembles tension-type headache in that it is accompanied by cervical dysfunction, and may have migraineous features.

Other modes for treating these various types of headaches include: (a) acupuncture, (b) biofeedback, and (c) chiropractic. However, studies have failed to show that any of these treatments is more effective than placebo. Acupuncture requires a highly-trained therapist. Biofeedback and particularly, training in muscular relaxation may be helpful for tension-type headache in selected individuals, but controlled studies have not demonstrated consistent success in the above conditions.

Atypical facial pain, which has recently been classified as facial pain by the International Headache Society, manifests itself as a relatively constant, mostly unilateral pain and is unrelated to jaw function. This condition is not associated with sensory loss or other physical signs, and radiographic and laboratory studies are uniformly negative. This condition may occur as a residual from relatively uncomplicated dental work, but usually the cause is unknown. Many neurologists regard this condition as psychogenic. Amitriptyline at bedtime and/or various analgesics and narcotics are used, but atypical facial pain responds poorly to all forms of medication.

Cervical muscle hyperactivity (spasm), is an extremely common condition with many causes, including trauma, tension, response to an inflamed or subluxed joint, arthritic changes, poor posture or work habits, systemic disease and adjacent pathology. Common treatment modalities are physical therapy, chiropractic, and medications including muscle relaxants, NSAID's, analgesics, and antidepressants. Often despite all efforts made to alleviate this type of spasm, the condition becomes chronic. When a cervical joint becomes subluxed, it causes pain and restricted motion, local inflammation, and adjacent muscle spasm. Manipulation to unlock the joint is indicated but muscle spasm must be reduced first. Conventional methods of treatment include heat, ultrasound, electrogalvanic stimulation and massage. All of these methods are obviously-time consuming. The relation between the condition of cervical muscle spasm and the previously described headaches and facial pain can be demonstrated by the fact that the headache and facial pain patients do not respond as well to treatment in the presence of significant cervical muscle spasm. This lack of response is related to the fact that head and neck are a single functional unit. Present headache theory recognizes the sympathetic nervous system influence on headaches. In cases of significant cervical spasm, the superior cervical ganglion is likely to be mediating the neurogenic inflammation.

The need for a more appropriate method of treating migraine, tension and post-traumatic headache, atypical facial pain, and cervical muscle spasm is apparent as the current methods of treatment are often ineffective. Treatment with pharmacological agents is associated with toxicity and must be used systemically over prolonged periods of time and often for decades. These agents further do not meet with patient acceptance or compliance. The conditions herein described represent a tremendous economic loss, considering the number of individuals afflicted, the time lost from work, as well as the inability to enjoy a normal pain-free life.

The inventor's research strongly suggests that this phenomenon occurs elsewhere and specifically in the previously described ipsilateral maxillary nerve plexus, and that it is also implicated in headaches (migraine, tension, post-traumatic headache) and atypical facial pain, and somewhat related to cervical muscle spasm. It is likely that edema secondary to local inflammation occurs in the maxillary nerve plexus microcirculation, caused by vasodilation and increased vascular permeability. This edema causes pressure on nerves which creates local tenderness. Because of this area's relative accessibility, the inventor has found it possible to consistently demonstrate several elements of local sterile inflammation: (1) normal appearing tissue, (2) consistent tenderness which is strongly related to symptoms, (3) increased local temperature, and (4) positive response to cold.

No obvious tissue pathology or periodontal condition was noted in examination of over 800 patients with the above conditions. Even periodontal lesions of the maxillary molars, present in a few of these 800 patients (n=6) were located much closer to the gingival line, and appeared unrelated to the maxillary tender zone in the area of the root apices.

In one procedure carried out by blinded, inexperienced observers, they correctly identified the symptomatic side in 178/200 (89%) of symptom-free migraine and tension-type headache patients by intra-oral palpation to determine the laterality of the maxillary tenderness. In studies conducted under the inventor's supervision, in over 90% of headache and facial pain patients observed by experienced investigators, ipsilateral tenderness, even in the symptom-free state, was found in patients presenting histories of such symptoms. It was found that bilateral symptoms produced bilateral tenderness, directly proportional to symptom severity. Tension headache usually occurs bilaterally, and these cases invariably demonstrated bilateral tenderness. The tender zone in patients with cervical muscle spasm was similarly symptom-related. In 36/38 tension headache patients experiencing unilateral symptoms a 94.7% correlation was found between laterality of symptoms and tenderness. The tender zone in patients with cervical muscle spasm was similarly symptom related.

In preliminary data analysis, 1026 of 1100 (93.2%) mostly asymptomatic migraine and tension-type headache patients exhibited intraoral tenderness in the maxillary molar periapical area, with laterality and degree of tenderness closely related to laterality and severity of symptoms. Temperature increase was also associated with intra-oral tenderness in symptomatic patients. On 40 patients during unilateral migraine or tension-type headache, the posterior molar apical areas were palpated and their temperature recorded with a long-stem laboratory thermometer. A model YS 1 (Yellow Spring Instrument Co.) 43TA tele-thermometer with #406 probe was used. The temperature was consistently higher (37 of 40) and the area more tender (39 of 40) on the symptomatic side. Tenderness and elevated temperature are signs of inflammation.

One of the inventors herein has invented methods for treating migraine, tension and post-traumatic headaches, atypical facial pain, and cervical muscle hyperactivity comprising the application of cold, through the use of cold or frozen water or saline (0-4° C.), cold metal or ice, to the area of tenderness associated with the plexus formed by the posterior and middle superior alveolar branches of the ipsilateral maxillary nerve, as well as to other branches of the trigeminal nerve (U.S. Pat. Nos. 5,527,351; 5,676,691; 5,693,077).

It is theorized that the lowered tissue temperature reduces excessive local blood flow (vasoconstriction) a secondary plasma extravasation (edema) and thereby reduces pressure on the maxillary nerve. Because the sympathetic division of the autonomic (involuntary) nervous system is closely related to the trigeminal system, prolonged application of cold also appears to affect the sympathetic outflow via the cervical ganglion. In the majority of patients with cervical symptoms and tenderness, a significant relaxation of symptoms, decreased cervical tenderness, and increased cervical range of motion during cold application was noted.

While the procedure has proven to be very effective in improving the conditions of patients suffering from the above conditions, the instrumental setup using cold water, saline, metal or ice, is not mobile or portable. There is also frequently limited availability of cold water, ice or saline. Obviously there is a need for improving this procedure by making it more portable and available.

The applicant has found that a thermoelectric cooler can be used for the cooling purposes in this medical procedure without the limitations of using cold water, ice or saline.

The thermoelectric coolers are solid state heat pumps working under the principle of Peltier effects. Such coolers consist of a number of p- and n-type semiconductor pairs (referred to as couples) connected electrically in series and sandwiched between two ceramic plates. During operation, electricity is passed through the junctions between the pairs of dissimilar metals. The atoms of the dissimilar metals have a difference in energy levels which results in a step between energy levels at each of the metals' junctions. As electricity is passed through the metals, the electrons of the metal with the lower energy level pass the first step as they flow to the metal with the higher energy level. In order to pass this step and continue the circuit, the electrons must absorb heat energy which causes the metal at the first junction to cool (cold side of the cooler device). At the opposite junction, where electrons travel from a high energy level to a low energy level they give off energy which results in an increase in temperature at that junction (hot side of the cooler device). The capacity of the cooler is proportional to the current and number of n- and p-type elements.

Peltier device has been used to change the temperature of some body parts for medical purposes. U.S. Pat. No. 6,629,990 teaches the use of a Peltier device for cooling a portion of a patient's brain for the treatment of movement disorder episodes. U.S. Pat. No. 6,682,524 teaches the use of a Peltier device for moderating skin temperatures for dermatological procedures. U.S. Pat. No. 6,741,895 teaches the use of Peltier device for stimulating the nerves of the vagina through the moderation of temperature. U.S. Pat. No. 6,811,551 teaches the use of a Peltier device that can be inserted into a patient's vein.

The applicant has found that a thermoelectric cooler can be used to cool the intra-oral tenderness area related to the various forms of ailments described earlier, requiring only a small portable device and electricity. Electricity (from AC, DC outlets or batteries) is more readily available than cold water, ice or saline in a typical setting, such as office, home or even outdoors. In addition, the temperature of the electrical cooling unit can be almost instantaneously changed in a wide range according to the user's input.

While all conceivable electrical instruments that possess cooling capacities can be used, Peltier device is most preferred, because it can be made compact in size and is free from the use of refrigerants or mechanical pumping systems.

This invention will be further explained in detail in reference to the figures wherein:

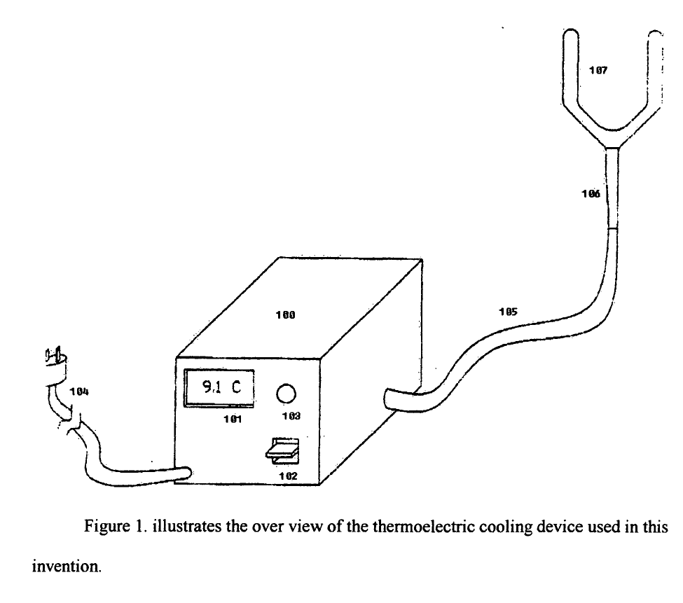

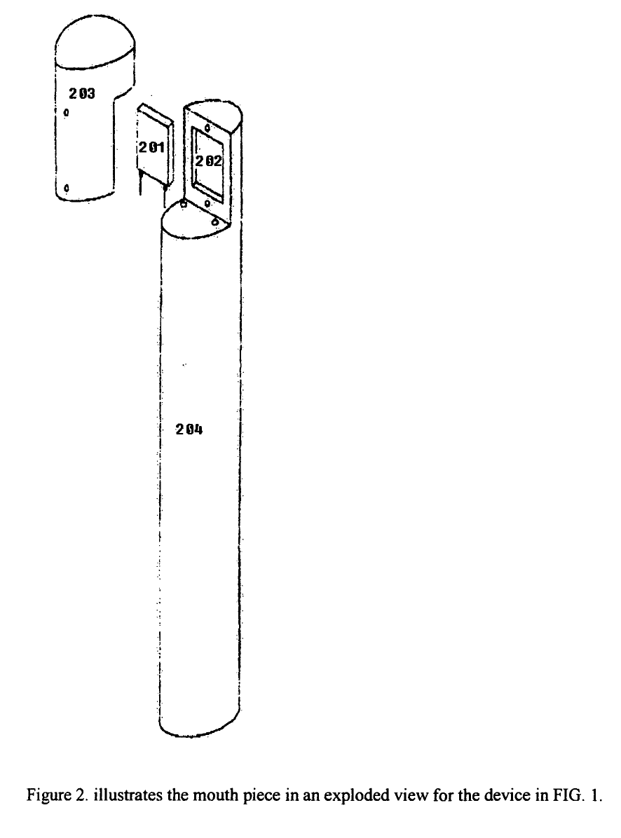

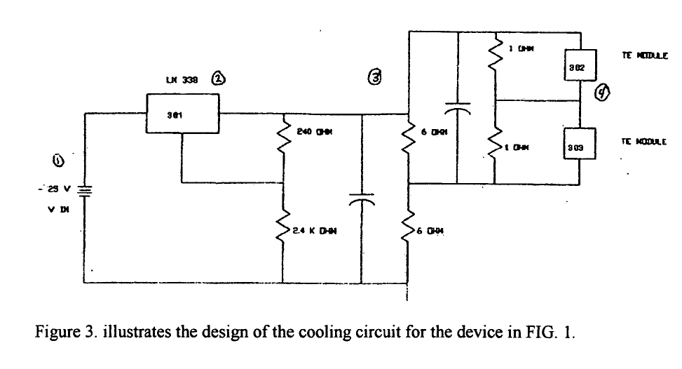

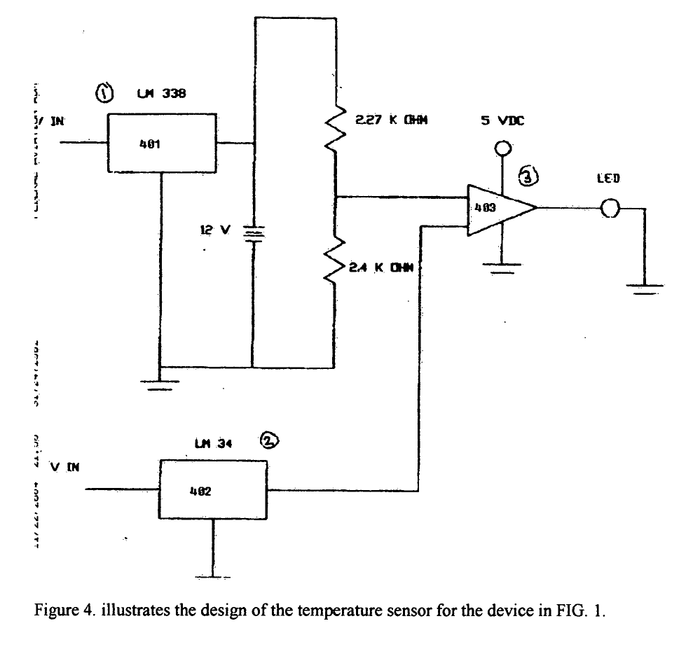

FIG. 1. illustrates the over view of the thermoelectric cooling device used in this invention.

FIG. 2. illustrates the mouth piece in an exploded view for the device in FIG. 1.

FIG. 3. illustrates the design of the cooling circuit for the device in FIG. 1.

FIG. 4. illustrates the design of the temperature sensor for the device in FIG. 1.

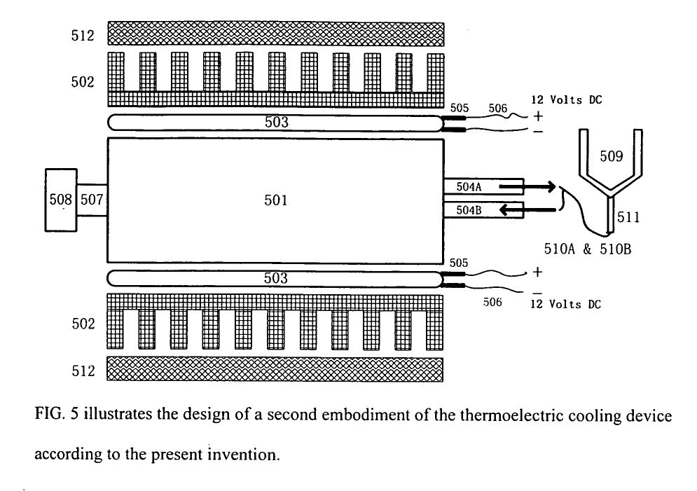

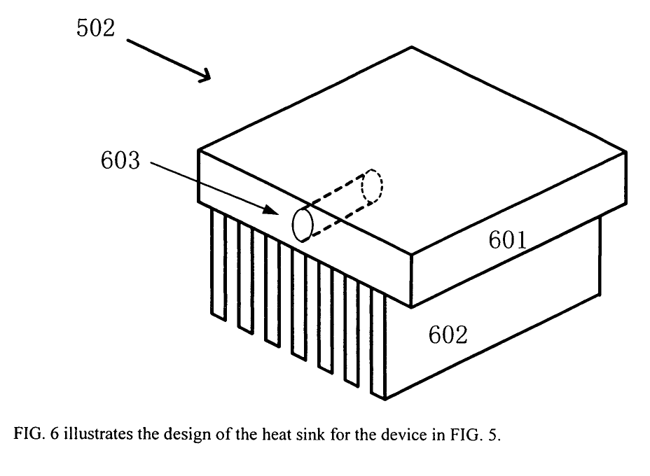

FIG. 5. illustrates the design of a second embodiment of a embodiment of the thermoelectric cooling device according to the present invention.

FIG. 6. illustrates the design of the heat sink for the device in FIG. 5.

The instant invention eliminates the use of cooling water, ice or saline by using a Peltier cooling unit.

A first and preferred embodiment of the portable cooling device for the intended medical procedure is shown in FIGS. 1-14.

FIG. 5. illustrates the design of second embodiment of the thermoelectric portable cooling device for the intended medical procedure.

As is shown in FIGS. 1-14. The first amendment comprises a controller unit 100 which contains an AC to DC converter and all controlling circuits. On the front of the controller unit, there are a temperature display 101, preferably in digital format, and on/off switch 102. An LED light is provided on the front of the controller unit 103, which when lit indicates that the measured temperature on the treated intra-oral zone is the same as the preset temperature. The device may further contain a temperature input means, for example in the form of a dial or buttons, allowing desired temperatures to be entered into the device. A power cord 104 is provided and attached to the AC/DC converter inside the controller for supplying AC power to the device. The device may further contain a battery compartment in the controller so that the device can be powered on batteries when AC power is not available. A wire harness 105 is provided and attached to the control circuits inside the housing. The wire harness is preferably flexible, for example, a gooseneck for easy operation. The wire harness houses five wires. Two wires are used to power the Peltier cooling modules. Another three wires transmit signals from the temperature sensor (voltage-in, voltage-out and ground). The distal end of the wire harness is attached, through a straight and preferably nonflexible portion acting as a handle 106, to the U-shaped mouth piece 107, whose two distal ends house the Peltier cooling units and temperature sensors.

The two prongs of the U-shaped mouth piece 107 are identical and they are explained in more detail in FIG. 2. A Peltier module 201 is contained in a recess 202 of each of the distal ends of the two prongs. A metal cover 203, shielded in rubber or plastic, is placed on top of the Peltier module forming a closed probe head. Thermal grease is preferably used on both sides of the Peltier module for more efficient heat transfer and screws can be used for assembling. The cover is in contact with the cold surface of the Peltier module 201. During application, the shielded cover 203 is in direct contact with the intra-oral point in the patient's mouth. The side of the Peltier module in contact with the recessed surface of the prong is the hot surface and the heat from the Peltier module is dissipated by the long prong stem 204. For effective heat dissipation and yet at the same time keeping the prong body from becoming too hot to hold, the prong stem 204, is most preferably made of metal tube encompassed in rubber or plastic shields. The rubber or plastic shields over the cover 203 and prong stem 204 further protect the patients from the risk of electrical shock. Inside of the metal tube of the prongs, there are circuits for supplying electrical power to the Peltier module and for taking temperature sensor readings. (where should the temperature sensor go?). The two prongs serve to treat two intra-oral points on both the left and right sides simultaneously if desired.

A preferred unit for use as the Peltier module is the Hot1.2-30-F2A series Thermoelectric Cooler (TEC) marketed by Melcor. It has the following specifications and capacities:

| | | |
|---|---|---|
| Hot side Temperature | 25° | C. |
| Qmax | 2.43 | watts |
| Delta Tmax | 64° | C. |
| Imax | 1.2 | Amps |
| Vmax | 3.6 | Volts |
| Module Resistance | 2.61 | Ohms |

FIG. 3. illustrates the cooling circuits for the device in FIG. 1. The AC/DC converter in the controller unit supplies DC current. Alternatively the power can be supplied by batteries housed in the controller unit or from external DC supplies. A voltage regulator LM338, a high current variable voltage-limiting device 301, is provided for regulating the DC input voltage over a broad range that is available in typical settings. For example, the voltage regulator is preferably set up and configured to allow an input voltage between twelve and twenty five volts DC, so that the device can be powered by the electrical outlets on most of the cars, boats and airplanes. The LM 338 is s product of National Semiconductor and is supplied by Digikey. The Plastic Package (TO-220). The resistor and capacitor combination of the circuit design will further limit the input voltage to a usable source. The power to the two thermoelectric modules 302 and 303 are supplied in parallel so that the voltage across each module is the same.

FIG. 4. illustrates the design of the temperature sensor. The voltage regulator LM338 401 performs multiple function is also used for supplying the power to the temperature sensor from a wide range of DC power inputs. The temperature sensing device LM34 402, housed in 203, is embedded in the distal end of the prong so that an accurate reading of the temperature of the location where the mouth piece is in contact with the intra-oral zone can be obtained. The LM34 converts the changes in temperature into an equivalent voltage output. This particular device is made by National Semiconductor and is distributed by Digikey. The Plastic Package (TO-92) is used for this application. For the particular model, one degree in temperature equals 0.07 volts DC either up or down depending on the direction of voltage change. The feedback is supplied to the controller unit for adjusting the electrical power supplied to the cooling unit, so that the temperature of the cooling surface can be adjusted to a preset level prescribed by the doctor or intended by the patient. A differential amplifier (comparator) 403 measures the output of the LM34 and compares the output to a determined voltage. When the determined voltage is achieved from the LM34 and it matches the stationary voltage, the comparator will produce an output voltage and turn on a Light Emitting Diode (LED) 103.

An alternative embodiment of the present invention is illustrated in FIGS. 5-6. In this second embodiment, a suitable liquid, such as water, chilled using a thermoelectric cooling unit is circulated through the mouthpiece to maintain a therapeutically effective temperature at the distal ends of the mouthpiece. A container 501 is provided as a reservoir with a mouth 507 and a cap 508. The cap 508 can be temporarily removed during maintenance to allow the water to be replaced. A conventional pumping means (not shown) is present to enable a continuous flow of water in the closed system during use, from the container 501, through the outlet 504A, through the flexible outgoing tubing 510A, through the handle 511 and the U-shaped mouthpiece 509, through the flexible returning tubing 510B, through the inlet 504B, and eventually back to the reservoir 501. At least one thermoelectric cooling unit 503 is present. The cold surface of the thermoelectric cooling unit is in contact with the container 501 to chill the water during operation. The hot surface of the thermoelectric cooling unit is in contact with heat dissipating units/heat sinks 502. The heat sink can take any conventional form, such as a cast iron liner 601 fitted with multiple aluminum fins 602 as shown in FIG. 6. The heat sink can further contain one or more sensors 603 therein or thereon to provide a temperature feedback to the controlling circuits (not shown). One or more electrically powered fans 512 can be provided to increase the airflow around the heat sink and thereby improve the heat dissipation efficiency. The thermoelectric cooling units 503 are powered by a DC supply (not shown) via pins 505 and electrical wires 506. This embodiment allows the operation of the device without requiring ice or other external cooling means as long as electricity is available. The use of water housed in a closed system removes the need for a continuous supply of water.

The desired temperature for the intended treatment procedure is in the range from 0 to 10° C.

The total weight of the device is between 0.5 and 2 pounds, most preferably between 0.5 and 0.75 pounds for portability.

When the device is to be used, it is first powered up by using either an AC or DC power source. A desired temperature is either preset or entered into the device using the provided input mechanism. When the temperature at the cold surface of the mouthpiece is the same at the set temperature, the LED light is turned on, indicating that the device is ready to be used. The user can hold the handle between the wire harness and the U-shaped mouthpiece, insert the distal ends of the mouthpiece into his/her mouth and hold the mouthpiece in its place for a period of time either preset or as medically necessary, while the cold surface is in contact with the intra-oral tenderness area on one or both sides. Typical length of time is 35 to 40 minutes. When the medical treatment is finished, the power is turned off. The mouthpiece is cleaned using conventional means and then dried.

In addition to using cold generated from the thermoelectric cooling device alone, anesthetics can be used in conjunction to provide enhanced relief Examples of such anesthetics include xylocaine, carbocaine, marcaine, xylocaine, epinephrine, neo-cobefrin, marcaine and citanest used alone or in combination. When epinephrine or an equivalent is present, vasodilatation is observed, thereby resolving the plasma extravasation (edema) which reduces pressure on the maxillary nerve as well as interrupting the pain cycle. In the majority of patients with cervical symptoms and tenderness, a significant reduction of cervical symptoms, increased cervical range of motion, and decreased posterior cervical electromyographic (EMG) signal reduction during the combined cold/anesthetic application was noted. It was additionally found that by increasing the cold application time to approximately 40 minutes, additional benefits were observed, even without addition of local anesthetic. Such benefits include:

1. Significantly better results in eliminating acute migraine and tension-type headaches, even if symptoms had lasted for several days. In the recalcitrant headaches, greater improvement was noted during minutes 20-40 of application, than in the first 0-20 minutes. Additionally, these acute headaches were less likely to return when the additional treatment time was utilized.

2. Some non-responsive cervical muscle spasm cases also responded positively with increased treatment time.

We claim:

1. A method for medical treatment of a condition selected from the group consisting of migraine, tension type and post traumatic headache, atypical facial pain, and cervical muscle hyperactivity comprising applying a cold supplying probe of a portable thermoelectric cooler to the zone of intra-oral tenderness located in the area of the plexus formed by the posterior superior alveolar branch of the ipsilateral maxillary nerve of a subject having one of the said conditions.

2. A method according to claim 1, wherein the method further comprises injecting a local dental anesthetic in a dental pain relieving amount into said zone of intra-oral tenderness in conjunction with the application of said cold supplying device.

3. A method according to claim 2, wherein the said local dental anesthetic is selected from the group of xylocaine, carbocaine, marcaine, xylocaine with epinephrine, carbocaine with neo-cobefrin, marcaine with epinephrine and citanest.

4. A thermoelectric cooling device for applying cold to a subject having a condition selected from migraine, tension type and post traumatic headache, atypical facial pain, and cervical muscle hyperactivity, comprising contacting the cold supplying probe with the zone of intra-oral tenderness located in the area of the plexus formed by the posterior alveolar branch of the ipsilateral maxillary nerve of said subject, said device comprising a controller unit, a wire harness, a handle, and a U-shaped mouth piece, each distal end of said mouth piece containing a thermoelectric cooling module and being capable of applying cold to the said intra-oral zone on at least one side of the subject's mouth.

5. A thermoelectric cooling device for applying cold to a subject having a condition selected from migraine, tension-type and post-traumatic headache, atypical facial pain, and cervical muscle hyperactivity, comprising contacting the cold supplying probe with the zone of intra-oral tenderness located in the area of the plexus formed by the posterior alveolar branch of the ipsilateral maxillary nerve of said subject, said device comprising a controller unit, a water container, tubing for allowing water flow out of and into said container, at least one thermoelectric cooling unit, at least one heat sink, a handle, and a U-shaped mouth piece, said thermoelectric cooling unit being situated between said container and said heat sink, the distal ends of said mouth piece being capable of applying cold to the said intra-oral zone on at least one side of the subject's mouth.
